(19) United States
(12) Patent Application Publication
(10) Pub. No.: US 2018/0052144 A1
(43) Pub. Date: Feb. 22, 2018

(54) Method And Technique For Verification Of Olive Oil Composition

(71) Applicant: Hormoz AZIZIAN, Oakville, ON (CA)

(72) Inventor: Hormoz AZIZIAN, Oakville, ON (CA)

(21) Appl. No.: 15/556,736

(22) PCT Filed: Jan. 26, 2016
(86) PCT No.: PCT/CA2016/000026

§ 371 Date: Sep. 8, 2017

Related U.S. Application Data
(60) Provisional application No. 62/131,790, filed on Mar. 11, 2015.

(51) Int. Cl. G01N 33/03; G01N 21/359

(52) U.S. Cl. CPC G01N 33/03; G01N 2021/3595; G01N 21/359

(57) ABSTRACT

A new rapid Fourier transform near infrared (FT-NIR) spectroscopic method is described to screen for the authenticity of extra virgin olive oils (EVOO) and to determine the kind and amount of an adulterant in EVOO. To screen EVOO, a partial least squares (PLS1) calibration model was developed to estimate a newly created FT-NIR Index based on the carbonyl overtone absorptions in the FT-NIR spectra of EVOO and other mixtures attributed to volatile (5200 cm⁻¹) and non-volatile (5180 cm″1) components. Spectra were also used to predict the fatty acid (FA) composition of EVOO or samples spiked with an adulterant using previously developed PLS1 calibration models and modified for this purpose. To identify the type and determine the quantity of an adulterant, gravimetric mixtures were prepared by spiking an EVOO with different concentrations of each adulterant. Based on FT-NIR spectra, four PLS1 calibration models were developed for four specific groups of adulterants each with a characteristic FA composition. These different PLS1 calibration models were used for prediction of type and amount of adulterant. A more comprehensive double or triple adulterant PLS1 calibration models were also developed and tested which provided means of analyzing commercial virgin olive oils which had been adulterated.

### FIELD OF THE INVENTION

The present invention relates to a method for measuring the purity of an organic food product, and in particular, relates to a method for the determination of the purity and freshness of olive oil samples using an FT-NIR spectroscopy based analytical technique.

### BACKGROUND OF THE INVENTION

Establishing the authenticity of food products, and in particular, extra virgin olive oil (EVOO) continues to be of great interest to scientists and consumers, and detecting adulteration of EVOO for economic gain is an ongoing concern for regulatory agencies. Adulteration of EVOO, involving the replacement of high cost EVOO with lower grade and cheaper substitute of can be very attractive and lucrative for a food manufacturer or raw material supplier. The adulteration of EVOO can also have major heath implications to consumers. As such, the detection of EVOO adulteration is of importance.

In olive oil production, there are various methods for extracting the oil that yield different quality grades. Extra virgin olive oil is obtained from the olive, Olea europaca, by purely mechanical mean. Lower grade refined oils, which will be referred to as “refined oils” are obtained from the olive by solvent extraction, heat treatment, esterification, or other refining techniques. The composition of the oils collected is based on the fatty acids present in the olive cultivar (of which there are hundreds). Moreover, the oil composition varies not only with the type of oil and extraction method but also with geographical origin and meteorological effects during the growth and harvest of the olives.

There are many olive oil standards that have been approved and published by various associations and countries that define grades of olive oils and specify chemical composition and quality parameters. These standards are regularly amended to accommodate the natural variations in olive oil cultivars and to upgrade them if new components are discovered in EVOO. These standards also typically include various analytical techniques that can be used to verify the grade and quality of the oil being tested. In fact, a variety of prior art physical and chemical tests have been used to establish the authenticity of olive oil and to detect the level of adulterants in it.

Many of the official methods used to detect adulteration of EVOO are labor intensive and time consuming. As one approach, the analysis of fatty acid profile of an oil, after methylation using gas chromatography (GC), has been reported for the quantification of seed oils in olive oil.

High Performance Liquid Chromatography (HPLC) analysis of the fatty acid and triglycerides composition has also been studied for detection of adulteration of olive oil. For example, Jabeur et al. (Jabeur, H.; Zribi, A.; Makni, J.; Rebal, A.; Abdeihedi, R.; Bouaziz, M.; “Detection of Chemlali extra-virgin olive oil adulteration mixed with soybean oil, corn oil, and sunflower oil by using GC and HPLC”; J. Agric. Food Chem. 2014, 62, 4893-4904.) recently applied gas chromatography (GC) and high performance liquid chromatography protocols to determine the minimum detectable quantities of soybean, corn, and sunflower adulterant oils in a specific EVOO cultivar by determining fatty acid (FA), triacylglycerol (TAG), and sterol compositions.

Nuclear magnetic resonance (NMR) analysis and a spectrofluorometric methods have also been reported for detecting the adulteration of olive oil, however, this approach is not typically feasible for rapid, routine sample testing.

Moreover, a drawback to et least some of these conventional methods of analyzing oils is that they are destructive and time-consuming, and/or involve the hydrolysis and methylation of the resulting fatty acids. As such, they are typically time consuming, and often provide little information on the composition of the EVOO sample being tested.

UV spectroscopy based on 208-210 and 310-320 nm has been widely used to detect the adulteration of extra virgin olive oil with refined olive oil. Unlike chromatographic procedures, vibrational spectroscopy techniques offers unique advantages because they are typically rapid, non-destructive, and can be applied to measure neat oils without any sample preparation or dilution in any solvent.

For example, Sinelli et al. (Sinelli, N.; Cerretani, L.; Di Egldio, V.; Bendini, A.; Casiraghi, E.; “Application of near (NIR) infrared and mid (MIR) Infrared spectroscopy as a rapid tool to classify extra virgin olive oil on the basis of fruity attribute intensity”; Food Res. Internat. 2010, 43, 369-375.) applied mid-Infrared (MIR) and near-infrared (NIR) spectroscopic techniques in conjunction with multivariate statistical methods to classify EVOO based on sensory attributes.

Rohman et al. (Rohman, A.; Che Man, Y. B.; Yusof, F. M.; “The use of FTIR spectroscopy and chemometrics for rapid authentication of extra virgin olive oil”; J Am. Oil Chem. Soc. 2014, 91, 207-213.) similarly applied multivariate calibration tools and MIR for the rapid authentication of EVOO, and also to classify EVOO adulterated with palm olein (Rohman, A.; Che Man, Y. B.; “Fourier transform infrared (FTIR) spectroscopy for analysis of extra virgin olive oil adulterated with palm oil”; Food Res. Internat. 2010, 43, 886-892.).

These techniques have been successfully used to quantify levels of walnut oil, refined olive oil (see Lai, Y. W.; Kemsley, E. K.; Wilson, R. H.: “Quantitative analysis of potential adulterants of extra virgin olive oil using infrared spectroscopy”; Food Chem. 1995, 53, 95-98.) and sunflower oil (see Tay, A.; Singh, R. K.; Krishnan, S. S.; Gore, J. P.; “Authentication of olive oil adulterated with vegetable oils using Fourier transform Infrared spectroscopy”; Lebensm.-Wiss. Technol. 2002, 35, 99-103.) in EVOO.

Internal reflection MIR spectroscopy and chemometrics were also used by de le Math et al. (de la Mata, P.; Dominguez-Vidal, A.; Bosque-Sendra, J. M.; Ruiz-Medina, A.; Cuadros-Rodriguez, L.; Ayora-Cañada, M. J.; “Olive oil assessment in edible oil blends by means of ATR-FTIR and chemometrics”; Food Control 2012, 23, 449-455.) to distinguish between various blends of olive oils, while Bendini et al. (Bendini, A.; Cerretani, L.; Di Virgilio, F.; Belloni, P.; Bonoli-Carbognin, M.; Lercker, G.; “Preliminary evaluation of the application of the FTIR spectroscopy to control the geographic origin and quality of virgin olive oils”; J. Food Quality 2007, 30, 424437.) investigated the capability to discriminate virgin olive oils based on geographic origin.

Visible and NIR spectra were applied to the classification of EVOO from eastern Mediterranean countries on the basis of their geographical origin.

Gore et al., in U.S. Pat. No. 7,288,768, also describes a technique for analysing olive oil using selected infrared frequencies.

Christy et al., (Christy, A. A.; Kasemsumran, S.; Du, Y.; Ozaki, Y.; “The detection and quantification of adulteration in olive oil by near-infrared spectroscopy and chemometrics”; Anal. Sci. 2004, 20, 935-940) describes a technique using FT-NIR (Fourier Transform-Near Infrared) to identify various components of adulterated olive oil. While measuring olive oil samples spiked with known concentrations of soybean, sunflower, corn, walnut, and hazelnut oils, Christy et al. reported very small differences in the FT-NIR spectra near a wave number of 5260 cm⁻¹. This and a second similarly weak band at a wave number of 6179 cm⁻¹ were attributed to 2nd overtones of the C═O stretching vibration. Based on the entire FT-NIR spectral range and partial least squares (PLS) calibration plots of oil mixtures these authors reported good correlation between the measured and predicted adulteration levels for a given EVOO spiked with one adulterant. Christy et al. also indicated that the models they created were only applicable to the specific pair of olive oil and adulterant investigated.

To date, however, there is no screening method that can rapidly authenticate EVOO, identify the nature of an adulterant in commercial EVOO products, and determine its concentration. As such, providing a rapid method for the authentication of the constituents of an EVOO sample would clearly be beneficial. This would be particularly advantageous if the analytical technique could be used to rapid verify the authenticity of an EVOO sample to verify that it was essentially pure EVOO, while being able to identify, and preferably quantify, 1 or more adulterants which might be present in the EVOO sample being tested.

As such, it would be advantageous to be able to provide a rapid technique for determining whether an EVOO sample had been adulterated, and if so, identify the type, nature, and amount of EVOO adulteration that had occurred.

### SUMMARY OF THE INVENTION

Accordingly, it is a principal advantage of the present invention to provide a method based on FT-NIR spectroscopy to rapidly analyse an edible oil sample, and preferably an olive oil sample, and most preferably an Extra Virgin Olive Oil (“EVOO”) sample, to determine whether the sample has been adulterated.

Moreover, it is a further advantage of the present invention to provide an FT-NIR spectroscopy based analytical technique of an olive oil, and preferably an EVOO, which can identify an olive oil adulterant, and preferably both identify and quantify, the level of adulteration, present in the olive oil sample.

It is a still further advantage of the present invention to provide an FT-NIR spectroscopy based analytical technique which can identify and quantify at least 1 adulterant present in an olive oil sample, and preferably an EVOO sample. More preferably, the FT-NIR spectroscopy based analytical technique is one which can be used to identify between 1 and 6 adulterants, and preferably between 1 and 3 adulterants, present in the oil sample being analysed.

The advantages set out hereinabove, as well as other objects and goals inherent thereto, are at least partially or fully provided by the method of the present invention, as set out herein below.

Accordingly, in one aspect, the present invention provides a method for the detection of adulteration in an edible oil, and preferably an olive oil sample, and most preferably, an EVOO sample, comprising:

preparing a calibration model for said edible oil, to be tested;

conducting an FT-NIR spectroscopy analysis of an unknown sample;

analysing the FT-NIR spectroscopy analysis of said unknown sample, at selected wave numbers; and,

comparing the FT-NIR spectroscopy analysis of said unknown sample, to said calibration model of said edible oil, at said selected wave numbers, in order to determine whether said edible oil sample had been adulterated.

In a preferred approach, a single wave number is used for comparison of the unknown sample, compared to the calibration model, and in a preferred embodiment, the selected wave number is at, or essentially at, 5280 cm⁻¹. By using the phrase “essentially at”, the skilled artisan will be aware that the band found at or near 5280 cm⁻¹, is to be analysed, and compared to the calibration model. In actual fact, the levels found for this band can be analysed over a wave number range of 5280+/−50 wave numbers, and more preferably over a range of 5280+/−15 wave numbers. In any event, it will be clear that the FT-NIR spectroscopy analysis peak of approximately 5280 cm⁻¹ is the FT-NIR spectroscopy peak of most interest in the present invention.

Thus, the present invention first provides a method using calibration models, based on analysis of the edible oil, and in particular, an EVOO, that have been developed for the observed FT-NIR spectra for the pure edible oil, or EVOO, in order to rapidly evaluate the authenticity of the edible oil, or EVOO product, and thus determine the presence of an adulterant in the edible oil, or EVOO product. The results observed at essentially 5280 cm⁻¹, or the two highly characteristic, weak and broad FT-NIR overtone features observed in the edible oil, and in particular, in EVOO, samples, which are essentially at or near 5180 and 5280 cm⁻¹, were used to calculate an FT-NIR index that served as a potential sensitive screening tool for authenticity of EVOO.

However, an important finding is the FT-NIR index, based on the results observed at the selected wave number (e.g. 5280 cm⁻¹), or ratio of selected wave numbers (e.g. 5280 cm⁻¹ and 5180 cm⁻¹) which show whether the low boiling (volatile compound) have been affected, or otherwise reduced. The index is at its highest (e.g. 100%) when pure EVOO is tested. Indices of over 80% are preferred, with levels of over 90, 95 or even more 99%, are more preferred. Once the index falls below these levels, adulteration of the oil sample is suspected.

Once adulteration of the edible oil sample, has been determined, in a further aspect, the present invention also provides a method for the determination of the type and/or quantity of the level of adulteration in an adulterated edible oil sample, and preferably an adulterated olive oil sample, and most preferably, an adulterated EVOO sample, comprising:

preparing an FT-NIR calibration matrix based on calibration models based on said edible oil, having at least one, and preferably up to 4, and even more preferably up to 6 adulterants;

analysing said adulterated edible oil using an FT-NIR spectroscopy based technique to produce an FT-NIR oil analysis;

comparing said FT-NIR oil analysis with said PT-NIR calibration matrix, to determine the type of adulterant, the level of adulterant present, or determine both the type and level of adulterant or adulterants, present in said sample.

Thus, preferably using the same FT-NIR spectra used to determine adulteration, the FT-NIR spectroscopy analysis can be compared to a calibration matrix that has been prepared based on the edible oil, and added adulterant. Calibration matrices can be prepared using the edible oil, and preferably, a selected EVOO, in combination with 1, 2, or more, adulterants, which are present at various levels. Using this analysis, the type and quantity of the adulterant(s) can be determined, and thus provide a qualitative and quantitative analysis of the sample to be tested.

In a preferred embodiment, the calibration matrices can be prepared using various adulterants of interest, and these include those adulterants which are commonly added to EVOO. Byway of example only, common adulterants include other oils, which typically include oils such as soybean oil, sunflower oil, corn oil, canola oil, hazelnut oil, high oleic acid safflower oil, peanut oil, palm olein oil, refined olive oil, caster seed oil, coconut, cotton seed oil, hemp oil, palm oil, palm kernel oil, poppy seed oil, rice bran oil safflower, sesame oil, and the like, and genetically modified oils, such as high olein canola, high oleic sunflower, high oleic soybean, and the like.

Combinations of 2 or more of these oils can also be added, however, typically, mixture of less than 5, and more typically 3 adulterant oils, are added to an EVOO. Generally, only one adulterant oil is added to an EVOO.

### DETAILED DESCRIPTION OF THE INVENTION

As background, it should also be noted that while there is no exact definition of the frequency range related to the term “near infrared”, generally, the term is used to define the range of frequencies between 4000 and 14000 cm⁻¹ wave number (2.5 to 0.71 microns), and the technique of the present invention is applicable over this general range. However, preferably, the FT-NIR technique of the present invention is practiced within the range of 4300 to 9000 cm⁻¹ (2.33 to 1.11 microns), and even more preferably, the technique is practiced within the range of 4500 to 9000 cm⁻¹ (2.22 and 1.11 microns).

Further, preparation of the calibration matrix is known to those skilled in the art, and may consist, at a simple level, as being a straight line comparison of the spectral data at a selected frequency to the spectral data obtained from the range of baseline materials. However, typically, the calibration matrix will be somewhat more complex mathematical model which can be used in order td compare a series of spectral data (e.g. frequency and transmittance and/or reflectance data). Using these mathematical models, a calibration matrix is prepared which is capable of determining the types and/or the amounts of a number of materials, such as fatty acids (FA) which may be present in a selected test material.

The mathematical models used to prepare the calibration matrix can be based on statistical analysis of the spectral data which have been compared to the other data, including gravimetric results, or results based on some other analytical technique, such as gas chromatography (GC); in order to analyse complex chemical mixtures and solutions.

Typically, the user will start by constructing a data matrix from, for example, the gravimetric or GC data, and FT-NIR spectra for a set of baseline materials. The calibration matrix is then prepared by mathematical analysis of the data matrix. Suitable mathematical approaches for preparation of the calibration matrix can include, for example, mathematical techniques such as multiple linear regression (MLR), principal component regression (PCR), and partial least squares regression (PLS), although other methods can be adopted.

The calibration matrix can be limited to only a selected type of material. As such, a less complex calibration matrix is required. However, as more types of materials are analysed or otherwise encountered, with different fatty acid types and with wider ranges of fatty acid levels, the calibration matrix will, by necessity become more complex. The skilled artisan, however, will be able to determine the complexity of the calibration matrix required for a selected application. As such, the skilled artisan would be able to select a calibration matrix “library” appropriate for the type of materials to be tested.

It should also be noted that even though the proposed FT-NIR analyses and analytical techniques, are presented and discussed in several and separate sections, they are preferably viewed as, and conducted as, a single analysis.

Each section of the analysis though, typically addresses approaches which are preferably based on specific PLS calibration models that are applied to the same FT-NIR spectrum obtained from a given EVOO product or oil mixture. Only when there is consistency between all sets of results, can one estimate with some confidence that a given test sample is authentic EVOO or a mixture of oils.

Generally, it is expected that once an EVOO has been detected, that a true EVOO will contain the full complement of numerous volatiles that give olive oils their characteristic aroma, and any reduction in these volatiles will reflect a decrease in the levels detected at or near the 5280 cm⁻¹ wave number, and thus, the FT-NIR index.

In addition, once an EVOO has been detected, the fatty acid (FA) composition of a sample EVOO oil can be measured, and this analysis should fall within the ranges that account for the genetic varieties of olive oils.

Finally, once adulteration of the EVOO has been determined, the analysis can be compared to various calibration models for the identification of the type and determination of the amount of an adulterant added to an EVOO product.

The FT-NIR protocol presented here is unique since it applies several different calibration models to the FT-NIR spectrum of a given test oil to determine whether it is in fact an EVOO. One could arrive at the same conclusion by using several analytical methods, but that process would be labor intensive and require expertise in a number of areas. On the other hand, the FT-NIR and PLS1 methodology is rapid and will provide the needed information on possible adulteration in minutes.

It should be noted that previously, the development of a reliable and rapid method to detect adulteration of EVOO was found to be challenging and generally considered to be extremely difficult using a single analysis. Chemical methods combined with chromatographic separations of fatty acid methyl esters (FAME) or triacylglycerol (TAG) are only effective to detect the presence of added edible oils to EVOO products provided the composition of the adulterated oil mixture is sufficiently different from that of EVOO, i.e., contain higher levels of 18:2n-6, 18:3n-6, and 16:0, lower levels of 18:1n-9 and 16:0, or possess a different TAG structure compared to that of EVOO. In such cases, it might be possible to detect a 10 or 20% addition. However, one could not detect the addition of a fully refined olive oil to an EVOO since both oils have the same FA and TAG compositions. For this reason non-FA components, such as sterols and aromatic compounds in EVOO have also been reported. In general the chemical/chromatographic procedures are time consuming, expensive, require expertise in different areas and special laboratory facilities. On the other hand, spectroscopic methods in combination with chemometric techniques are rapid and non-destructive.

In the present invention, a methodology for the rapid determination of authenticity of EVOO solely based on novel and complementary FT-NIR and preferably PLS1 (multi-component systems analyzed for each component separately) procedures, is provided. In the current study, rapid (2-5 min) FT-NIR and PLS1 procedures are conducted (see below) which are capable of determining the authenticity of an EVOO by measuring an FT-NIR “Index”, the FA composition of the EVOO or oil mixture, and the nature and concentration of an adulterant oil in EVOO. All this information is obtained from a single FT-NIR measurement using the appropriately developed PLS1 calibration models. Each of these individual measurements will be discussed in turn.

As such, the various features of novelty which characterize the invention are pointed out in the following discussion and examples. For a better understanding of the invention, its operating advantages and specific objects attained by its use, reference should be made to the accompanying tables, drawings, examples, and descriptive matter in which there are illustrated and described preferred embodiments of the invention.

### EXAMPLES

The novel features which are believed to be characteristic of the present invention, as to its structure, organization, use and method of operation, together with further objectives and advantages thereof, will be better understood from the following examples in which a presently preferred embodiment of the invention will now be illustrated by way of example only.

It is expressly understood, however, that the examples are for the purpose of illustration and description only and are not intended as a definition of the limits of the invention.

### BRIEF DESCRIPTION OF THE DRAWINGS

Analysis of the results of the experimental results, are provided in the accompanying drawings in which:

FIG. 1 is an FT-NIR absorption spectrum of extra virgin olive oil showing the presence of bands at 5180 and 5280 cm⁻¹;

FIGS. 2a, 2b, and 20 are spectrums showing a reduction of band intensity near 5280 cm⁻¹ in EVOO as a result of heating the oil at 50° C. for 10 min (2a), as a result of applying a vacuum at 260 mBar for 50 min (2b), and as a result of bubbling nitrogen gas through the oil for 50 min (2c);

FIGS. 3a and 3b are spectrums showing a reduction of band intensity near 5280 cm⁻¹ in EVOO as a result of mixing with refined olive oil (a) and with refined corn oil (b);

FIG. 4 shows spectral changes in the 5380-5780 cm⁻¹ range observed upon addition of increasing amounts of refined corn oil to EVOO;

FIG. 5a and SI) show the PLS1 predicted concentrations for (a) soybean oil and (b) palm olein adulterants in EVOO compared to gravimetrically added amounts;

FIG. 6 shows a PLS1 predicted concentrations for soybean oil adulterant in EVOO as a function of the gravimetrically added amounts using calibration Models LA (linoleic acid; solid squares), OA (oleic acid; solid triangles), and PD (palm olein; stars), wherein the predicted FT-NIR Index values (open circles) are also shown;

FIG. 7 shows PLS1 predicted concentrations for hazelnut oil adulterant in EVOO as a function of the gravimetrically added amounts using calibration Models LA (linoleic acid; solid squares), OA (oleic acid; solid triangles), and PO (palm olein; stars), wherein the predicted FT-NIR Index values (open circles) are also shown;

FIG. 8 shows PLS1 predicted concentrations for palm olein adulterant in EVOO as a function of the gravimetrically added amounts using calibration Models LA (linoleic acid; solid squares), OA (oleic acid; solid triangles), PO (palm olein; stars), and RO (refined olive oil; solid circles), wherein the predicted FT-NIR Index values (open circles) are also shown;

FIG. 9 shows PLS1 predicted concentrations for refined olive oil adulterant in EVOO as a function of the gravimetrically added amounts using calibration Models LA (linoleic acid; solid squares), OA (oleic acid; solid triangles), PO (palm olein; stars), and RO (refined olive oil; solid circles), wherein the predicted FT-NIR Index values (open circles) are also shown; and

FIG. 10 shows the effect of adding known amounts of soybean oil to a commercial EVOO suspected of being adulterated with an adulterant from the linoleic acid (LA) group.

### DETERMINING THE AUTHENTICITY OF AN EVOO BY ESTIMATING THE FT-NIR INDEX

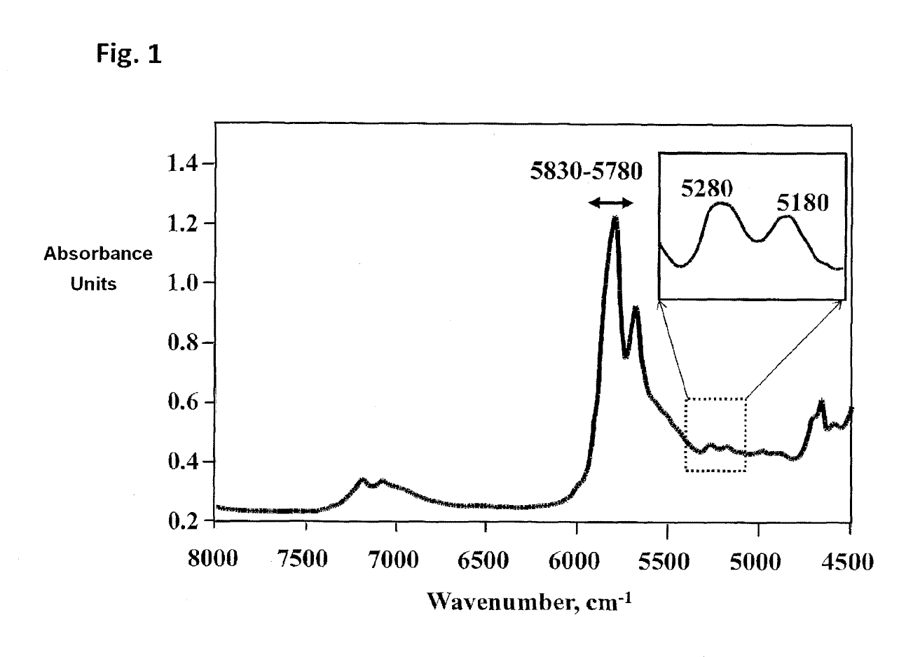

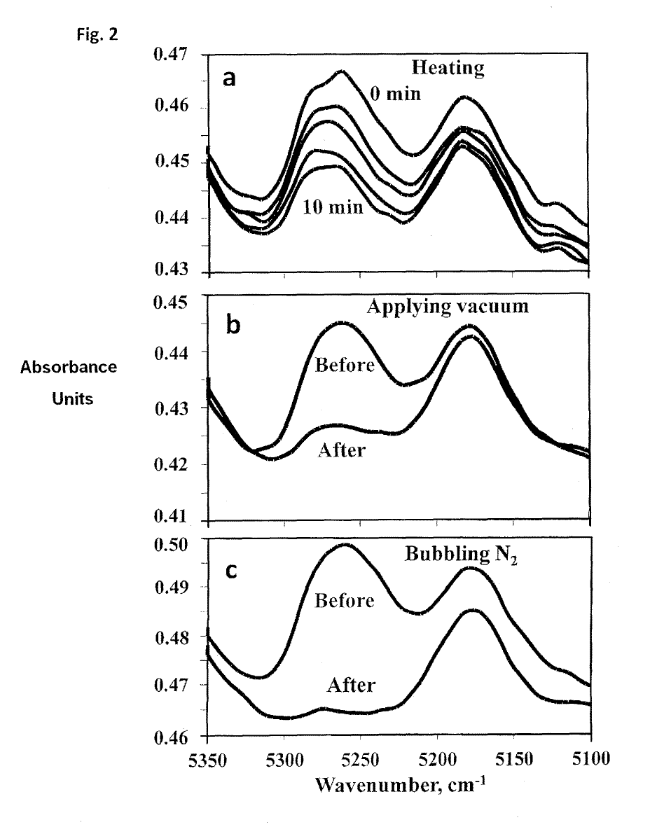

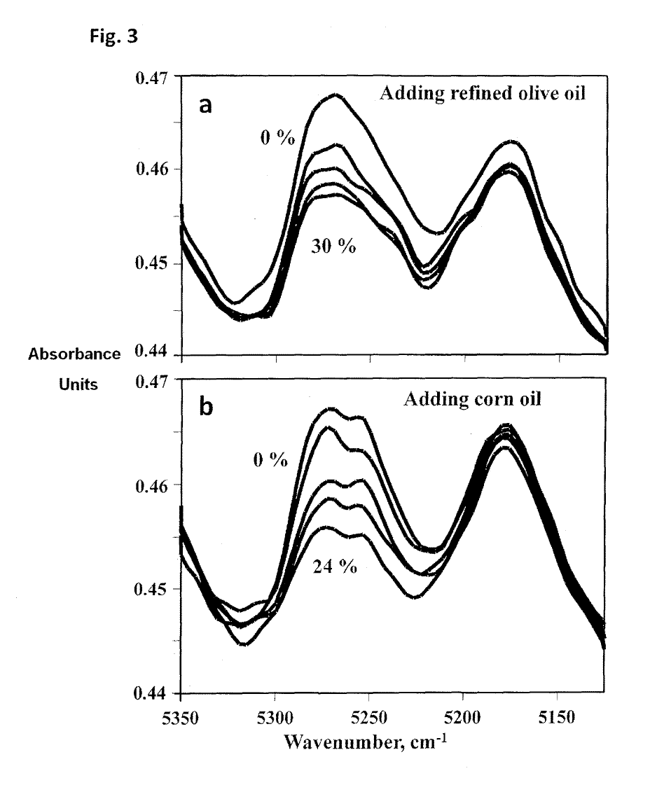

The authenticity of EVOO was evaluated after generating a PLS1 calibration model primarily based on the two weak, but highly characteristic FT-NIR absorption bands observed near 5180 and 5280 cm⁻¹. These two absorption band areas are in the carbonyl overtone region of the FT-NIR spectrum which was also reported by Christy et al., and are shown in FIG. 1. These two absorption bands are envelopes composed of a cluster of minor overtone bands as evident in FIGS. 2 and 3. In the current study we demonstrate for the first time that the band near 5280 cm⁻¹ observed for EVOO is mainly attributed to volatile compounds with carbonyl functional groups such as aldehydes, while the absorption band near 5180 cm⁻¹ consists of non-volatile carbonyl type compounds or esters. It is well known that volatile compounds are present in EVOO, and preliminary investigations of the volatile compounds in EVOO using gas chromatography/mass spectrometry (GC/MS) showed the presence of numerous aldehydes and other carbonyl-containing constituents.

In general, the intensity of the band near 5280 cm⁻¹ in authentic EVOO is relatively much greater than that near 5180 cm⁻¹. On the other hand, all the other edible oils, palm olein, and fully refined olive oil also show this absorption band near 5280 cm⁻¹, but at a much lower intensity relative to the one near 5180 cm⁻¹. The intensity of the band near 5280 cm⁻¹ in an EVOO can be reduced by heating the oil (FIG. 2a), applying vacuum (FIG. 2b), bubbling nitrogen through it (FIG. 2c), or by adding any of the other potential adulterant edible, fully refined oils characterized by a much lower absorption near 5280 cm⁻¹ (FIG. 3).

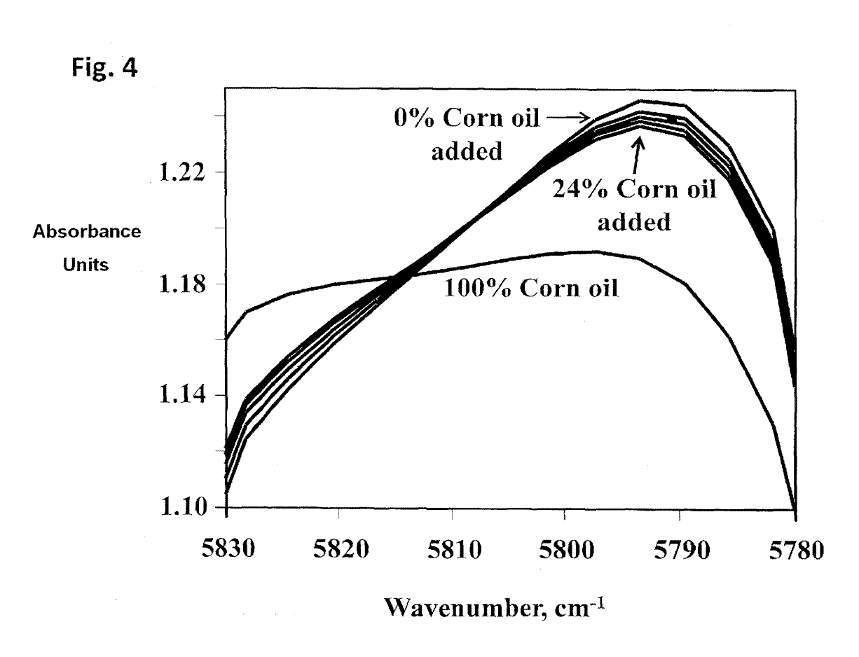

However, the intensity of the second band near 5180 cm⁻¹ does not change under any of these conditions and is similar for both EVOO and non-olive oils. The ratio of these two absorption bands (5280 cm⁻¹/5180 cm⁻¹) and other characteristic spectral regions, such as the overtones observed in the range 5830-5780 cm⁻¹ (FIG. 4), were used in the development of the PLS1 calibration model to determine a FT-NIR Index for any given olive oil or adulterated blend.

It is noted that some prior art documents have suggested that the absorption band near 5180 cm⁻¹ could be due to the O—H stretching vibration of water in other mediums. However, attributing the main absorption band near 6180 cm⁻¹ in the spectra of EVOO to water would seem highly unlikely since that would not account for its lack of susceptibility to moderate heat and vacuum. In fact, the intensity of the band near 5180 cm⁻¹ remained the same under all the conditions which affected the band at 5280 cm⁻¹. An attempt to dissolve water into EVOO failed to affect the intensity of the band near 5180 cm⁻¹; the drop of water simply sank to the bottom of the test tube. This would suggest that the band near 5180 cm⁻¹ is more likely due to non-volatile carbonyl or ester constituents that are common to all oils.

The presence of the minor absorption feature near 5280 cm⁻¹ in all the fully refined vegetable oils requires an explanation. This was rather surprising to observe that heating fully refined hazelnut oil for 10 min at 50° C. also resulted in a minimal reduction in the intensity of the significantly weaker band near 5280 ore, while the one near 5180 cm⁻¹ remained unchanged (data not shown). It would appear unlikely that any volatile components with carbonyl absorption could have survived the deodorization step during the refining stage of vegetable oils. The observed minimal reduction in intensity was most likely due to the loss of carbonyl-containing products of oxidation formed during storage or during the refining process. Unfortunately, FT-NIR spectroscopy cannot distinguish between naturally occurring volatile carbonyl type compounds in edible oils and those derived from subsequent oxidation.

The estimated FT-NIR Index value is only a rapid screening tool that reflects the condition of any edible oil at the time of measurement, and cannot determine the prior history of the oil and establish whether it was previously heated, refined, oxidized, or mixed with other oils. Based on the limited number of authentic EVOO and oil mixtures investigated in the present study, a FT-NIR Index value of less than 80 would require further investigation.

Determining the FA Composition of EVOO and other Edible Oil or Oil Mixtures

A major advantage of using FT-NIR spectroscopy is that the same FT-NIR spectra used to evaluate the authenticity of EVOO by estimating the FT-NIR Index are also used to determine the FA composition of the oil or oil mixture in question. PLS1 calibration models based on FT-NIR spectral differences have been previously shown to be capable of being successfully used to determine the FA composition of edible oils. Examples of the levels of fatty acids present in a variety of edible oils is shown in Table 1.

However, in the application of the present invention, spectral changes were clearly evident in the carbonyl (4900-5350 cm⁻¹; FIGS. 2 and 3) and the 1st C—H overtone region (5830-5780 cm⁻¹; FIG. 4). These spectral differences are due to both the FA and non-FA minor constituents present in these mixtures because FT-NIR spectroscopy is matrix dependent. The FT-NIR models can be optimized to rapidly determine the FA composition of all edible oils and oil mixtures investigated (see for example, the results shown in Table 2).

From the FA profile of an oil sample one may conclude adulteration if the FA composition is different from that characteristically associated with olive oils, provided sufficient amount of an adulterant is added. GC analysis of fatty acid methyl esters (FAME) was used to detect the presence of increased levels of soybean oil to EVOO. It should be noted that the FT-NIR method and PLS1 methodology is able to provide the same FA information at a fraction of the time and without the need of chemical conversion of the oils to FAME.

However, detecting adulteration of a commercial EVOO product by comparing only the FA might be limited. For example, one could detect an increase of 18:2n-6 provided sufficient amounts of oils high in 18:2n-6 are added, such as the common soybean, corn, sunflower and canola oils; see Table 2. However, when these oils are genetically modified to high OA oils, the 18:2n-6 marker disappears.

Detecting adulteration by observing changes in the levels of saturated FA in EVOO is also problematic and generally possible only when more than 20% of an adulterant is added such as hazelnut with a lower, and palm olein, with a higher content of 16:0 (Table 2).

Moreover, the more common and least detected adulterant, refined olive oil, is impossible to detect by comparing the FA or TAG composition. For this reason alternative methods are required, such as the method of the present invention. The benefit of the present FT-NIR spectroscopy and PLS1 methodology is related to its matrix dependency that reflects the presence of all matrix components in an EVOO product and thus allows the identification of oil matrices based on their differences.

### Materials and Methods

Two reference olive oils were purchased from Sigma-Aldrich Co. (St Louis, Mo., USA), and according to the label both were from Tunisia: namely EVOO (product number W530191) and refined olive oil (product number O1514). Several authentic EVOO samples were provided by the California Olive Ranch (Oroville, Calif., USA). Commercial EVOOs were also locally purchased. Refined vegetable oils were obtained from local grocery stores, while a palm olein sample was purchased from a local store in Indonesia and another product from Thailand was obtained from amazon.com.

During testing, all spectra were obtained using Bruker Optics (Billerica, Mass., USA) FT-NIR spectrometers, model Matrix F or MPA, equipped with a diffuse reflection fiber optic probe and with a liquid attachment. After each measurement, the probe was cleaned with a dilute (5% v/v) aqueous solution of a dish liquid detergent, rinsed with water, and dried.

All PLS1 calibration models were generated (using a technique developed by NIR Technologies Inc., Oakville, Ontario, Canada) by using Bruker OPUS software. The FT-NIR spectrometers were equipped with a thermoelectrically cooled InGaAs detector. Except for temperature controlled experiments, all spectra were collected at room temperature using 8 cm⁻¹ resolution and the Blackman-Harris 3-term apodization function. Test oils were placed in 10-ml beakers or a custom-made non-disposable test tubes designed to fit the FT-NIR probe attachment. The absorption spectra were subsequently collected. Six replicate absorption spectra were measured for each test portion. These spectra were subsequently used to generate an average spectrum and in the development of PLS1 calibration models for the determination of FT-NIR Index values, FA composition, and adulterant type and concentration in spiked EVOO samples.

Results—Inspection of Relative FT-NIR Band Intensities Near 5180 cm⁻¹ and 5280 cm⁻¹

The FT-NIR absorption spectrum of an EVOO exhibited two characteristic minor broad bands near 5180 and 5280 cm⁻¹ (FIG. 1). On repeated measurements of the same EVOO portion over an hour at room temperature a decrease of the band near 5280 cm⁻¹ relative to the one near 5180 cm⁻¹ was observed. To better characterize this change in intensity a sample of EVOO was heated at 50° C. for about 10 min and spectral acquisitions were simultaneously carried out throughout this time (FIG. 2a). To determine whether the decrease in the absorption band intensity near 5280 cm⁻¹ was related to the loss of some volatile components in EVOO, a vacuum at 260 mBar was applied to an EVOO test sample at ambient temperature for 50 min, and the oil was subsequently rescanned by FT-NIR spectrometer. A significant decrease in intensity near 5280 cm⁻¹ was observed (FIG. 2b). A similar result was observed after bubbling nitrogen gas through EVOO at room temperature for 50 min (FIG. 2c). There was also a reduction in the 5280 cm⁻¹ band intensity by adding either fully refined olive oil (FIG. 3a), corn oil (FIG. 3b), or other fully refined vegetable oils or palm olein (not shown) to an EVOO.

The second weak band near 5180 cm⁻¹ behaved differently and showed no significant susceptibility to heat, vacuum, bubbling with nitrogen gas or addition of refined oils; it remained essentially unchanged. In an attempt to test the hypothesis that the bands near 5180 cm⁻¹ and 5280 cm⁻¹ may be due to the O—H stretching vibration in water, 10 μL of water was added to an EVOO, however, no spectral change was observed under these experimental conditions.

### Development of a PLS1 Calibration Model to Generate a FT-NIR Index

A PLS1 calibration model was developed using the two weak, but highly characteristic FT-NIR overtone bands near 5180 and 5280 cm⁻¹ (FIGS. 2 and 3) plus other spectral regions that showed spectral differences (FIG. 4) to generate a FT-NIR Index. All test samples used in the development of a PLS1 calibration model were scanned as stated above. They included EVOO from known sources, EVOO spiked with refined olive oil or potential adulterants, and those that were treated with heat, vacuum, or nitrogen gas as described above.

The FT-NIR index was created primarily based on the ratio (5280 cm⁻¹/5180 cm⁻¹) of the integrated band areas for all the test samples. To eliminate the need to subsequently measure and integrate the area of these two absorption bands for future unknown test oils, a PLS1 calibration model was created. To generate this PLS1 model, several spectral ranges were used which included the two characteristic features near 5180 and 5280 cm⁻¹. Based on the highest normalized ratio (approximately 1.7:1.0) found for these two integrated band areas the analyzed EVOO product was arbitrarily assigned a FT-NIR Index value of 100; all other oils had lower values on this arbitrary scale. For instance, the FT-NIR Index values for the EVOOs from the California Olive Ranch ranged between 92 to 100 on this scale when they were analyzed upon receipt, while the certified EVOO and refined olive oil reference sample from Sigma-Aldrich had values of 90 and 40, respectively. The values for EVOO samples labelled as imported from Italy ranged from 86 to 97.

To expand the scale of the FT-NIR Index to near zero and to estimate more accurately the lower end of the FT-NIR Index scale, a synthetic triolein (Nu Chek Prep Inc., Elysian, Minn., USA) was used which showed an extremely low band ratio of 0.09 corresponding to a KT-NIR index value of 5. A constant offset elimination preprocess was applied in the PLS1 analysis. The coefficient of determination (R2) was 99.5 and the root mean square error of cross validation (RMSECV) was 1.7.

### Development of PLS1 Calibration Models to Rapidly Determine FA Composition

The previously developed PLS1 calibration models were based on observed FT-NIR spectra and used accurate GC determinations as primary reference. The FA calibration models used were optimized for the determination of FA markers in EVOO. More importantly, refinements were specifically made in the creation of individual FA models for the determination of FA markers in the present study, namely 16:0, 18:0, oleic acid (OA; 18:1n-9), linoleic acid (LA; 18:2n-6), and linolenic acid (LNA; 18:3n-3). First derivative and vector normalization preprocessing steps were applied for all FA except 18:2n-6 whose spectra were only vector normalized. The coefficient of determinations (R2) and the root mean square error of cross validation (RMSECV) were obtained for each of the FA, and they are respectively 16:0 (97.8, 0.8), 18:0 (96.2, 0.4), 18:1n-9 (99.7, 1.3), 18:2n-6 (99.2, 1.3), and 18:3n-3 (95.3, 0.5).

Gravimetric mixtures of authentic EVOO with the adulterants selected for testing were prepared (Table 1), and they ranged in concentration from about 3 to 30% of total weight, and in the case of refined olive oil to 60% of total weight (Table 2). These ranges were chosen based on the suspected adulterations of interest for economic gains. FT-NIR spectra were measured for each mixture. The changes in the FA composition of the mixtures increasingly reflected the contributions of the adulterant oils. For example, the evidence of adding oils high in 18:2n-6, such as soybean oil, was already evident at about 9-12% of total FA showing an increase in 18:2n-6 (and 18:3n-3) and a decrease in 18:1n-9 in the mixtures (Table 2). The situation was very similar for canola, corn and sunflower oils where changes in the FA composition were consistent with the levels of FA present in these oils (Table 2). The addition of oils with high levels of 18:1n-9, such as hazelnut, high QA safflower, and peanut oils were more difficult to detect.

However, it is possible to detect these oils at a level of about 20% mainly because of a lower content of 16:0 and a slightly higher content of 18:2n-6 (Table 2). Blends of palm olein and EVOO are also challenging to detect because of their fairly similar FA profile, but the addition of palm olein may be identified by an increase in 16:0 at about 8% adulteration (Table 2).

Mixtures of refined olive oil with EVOO are impossible to detect based solely on the FA profile, because the loss of volatiles during refining of olive oils does not change the FA composition. It is therefore apparent from these results that a comparison based on the FA composition of mixtures of EVOO and refined oils is limited in value, and with refined olive oil is not possible. Moreover, one should keep in mind that some commercial vegetable oils (such as soybean oil) may be genetically modified (high oleic acid soybean oil) and without proper label declaration, and thus would have a very different FA profile.

### Development of PLS1 Calibration Models to Determine the Type and Amount of Adulterant

To determine which adulterant was mixed with EVOO required a new approach that was not based on the FA composition established by GC as primary reference, but on gravimetrically prepared mixtures of these oils. To accomplish this task, EVOO was spiked with each of the adulterants listed in Table 1 in incremental levels as stated above (Table 2). It was not possible to generate a single PLS1 calibration model for all these adulterants. However, it was noticed that spectral similarities between certain types of adulterants, and therefore the adulterant oils were sorted into four distinct groups based on their characteristic FA profiles.

The first group consisted of adulterants containing high levels of LA, such as soybean, sunflower, corn and mole oils from which Model LA was generated. The second group consisted of adulterants with high levels of QA such as hazelnut, high OA safflower, and peanut oils which were used to generate Model OA. Groups three and four were specific for palm olein and refined olive oil from which Model PO and Model RO were developed, respectively.

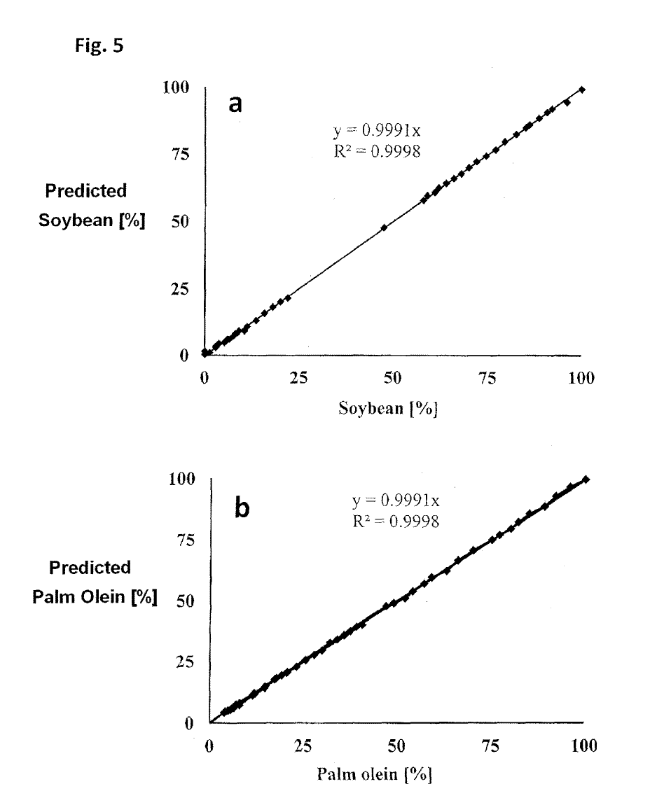

A constant offset elimination preprocessing step was therefore applied in all but the PLS1 model for OA which required only a straight line subtraction. The R2 and RMSECV for each model were: LA (99.9, 0.9), OA (99.5, 2.2), PO (99.9, 1.0) and RO (97.6, 3.7). All four models showed a significant linear regression correlation between the PLS1 predicted values based on FT-NIR data compared to the gravimetrically established concentrations of oil added to EVOO; data for soybean oil (FIG. 5a) and palm olein (FIG. 5b) are shown as examples.

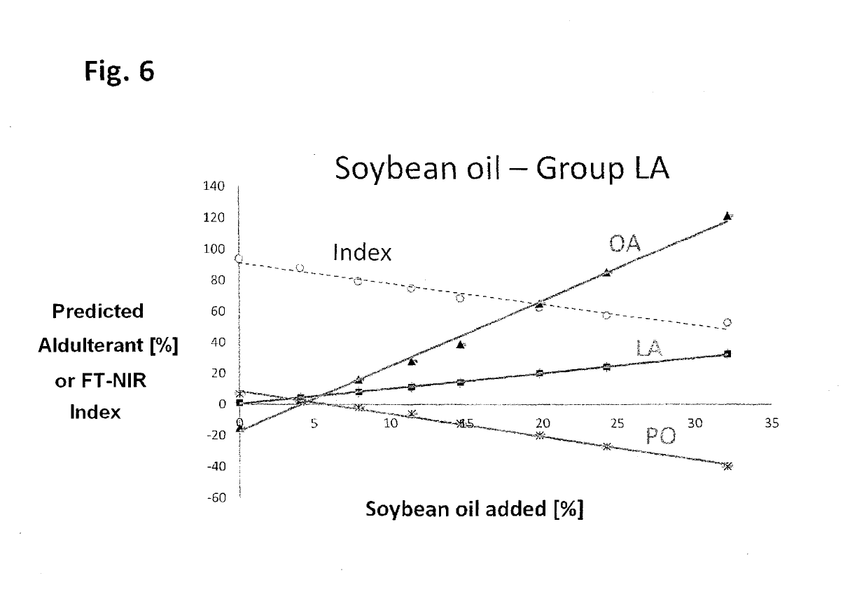

After generating the four PLS1 calibration models, the oils in each group were analyzed using all these models. The predictions of all FT-NIR measurements using all four calibration models yielded sets of linear correlation functions each with unique slope patterns associated with increased concentration of an adulterant (Table 3). For instance, increased spiking of EVOO with soybean oil showed the following slope pattern: a small positive slope with Model LA and a much higher slope with Model OA, while the slope obtained by using Model PO was negative (FIG. 6). The correct prediction model should yield a zero or negligible intercept, while a larger positive or negative intercept is an indication that the model used for prediction is the incorrect model. Spiked samples with the edible oils of sunflower, corn and canola showed similar slope patterns (not shown) (Table 2).

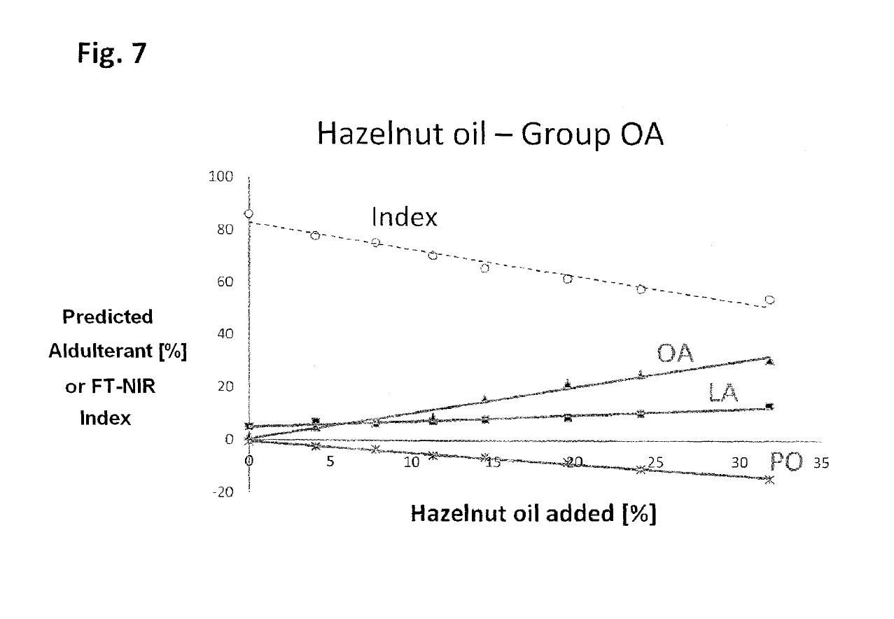

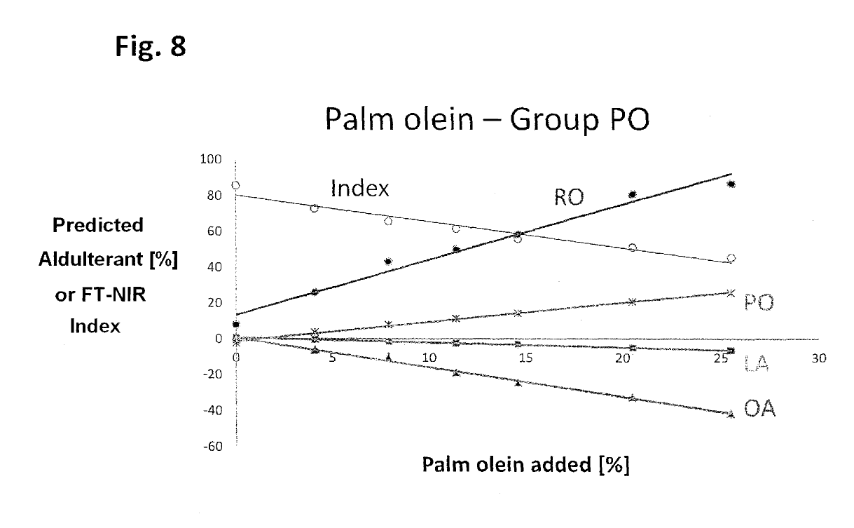

The FT-NIR Index was included in FIG. 8 to show how the addition of soybean oil also reduced the FT-NIR index that served as a sensitive marker and screening tool for potential adulteration. Spiking with hazelnut oil is an example of the OA group, and it showed the following elope pattern: a small positive slope with Model LA and a slightly larger positive slope with Model OA, while the slope obtained by using Model PO was negative (Table 3; FIG. 7).

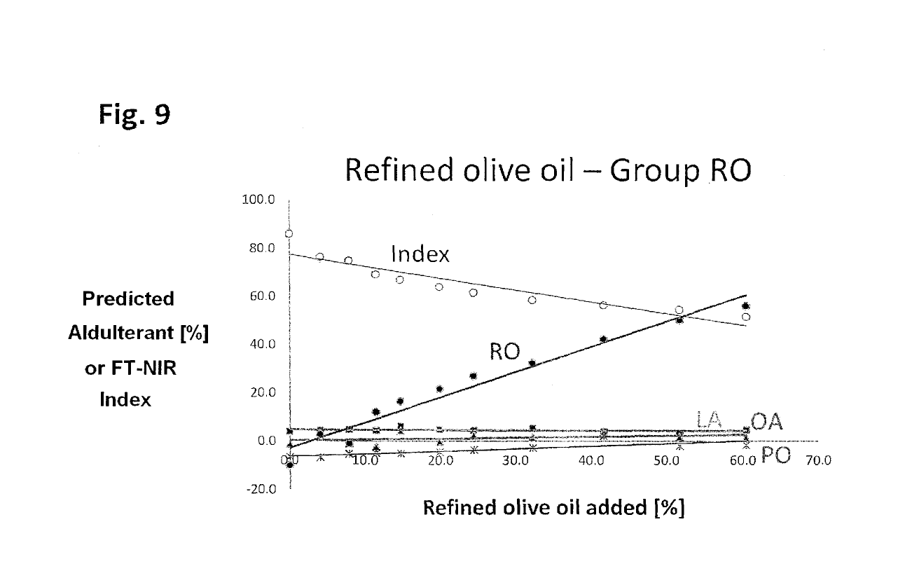

Again the FT-NIR Index decreased with the addition of hazelnut oil. The pattern of unique set of slopes applied also to palm olein and refined olive oil that showed specific responses unique to these two oils (Table 3). In the case of palm olein, Model RO slope was positive and greater than that of Model PO, and both slopes for Model LA and Model OA yielded negative values (FIG. 8). On the other hand, adulteration with refined olive oil showed a significant increase in the slope for Model RO but very low slope values for the other three models (FIG. 9). These four unique trends in slopes were subsequently used as rules to determine the nature of an adulterant and to predict the concentration of an adulterant in EVOO (Table 3).

However, if the prediction of whether an adulterant belongs to the LA or DA group is not clear, then the standard addition technique will be necessary to confirm the predicted identity and amount of an adulterant in EVOO, as discussed below.

### Confirming Adulterant Type and its Amount Using the Standard Addition Technique

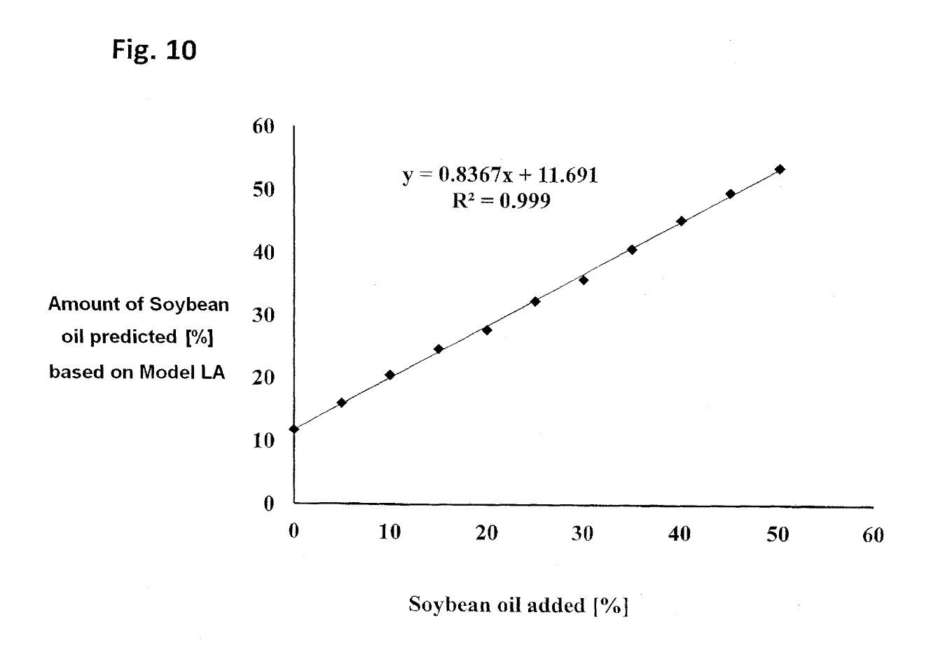

A standard addition technique was previously used to quantify low trans FA levels in fats and oils and was used in this study to confirm the findings of the PLS1 calibration models with respect to the type and amount of adulterant in EVOO. To demonstrate the potential of standard addition, a commercial olive oil sample was selected that had a low FT-NIR Index, which suggested that the oil was probably adulterated. Based on the spectral analysis using all four PLS1 calibration models, adulteration with an oil high in LA was suspected. This experiment indicated that Model LA would yield the correct identification and quantification (11.7%) of the suspected adulterant. This test sample was then spiked with several incremental amounts of accurately weighed soybean oil. The resultant FT-NIR standard addition measurements were analyzed with the PLS1 calibration Model LA (FIG. 10). The plot of predicted concentrations of adulterant in these mixtures after the gravimetric additions of soybean oil to the test sample resulted in a linear regression line (FIG. 10) that yielded a Y-intercept of 11.7% (at X=0) which confirmed that this EVOO product was adulterated with a high LA oil at that concentration.

### Determining the Type and Amount of Adulterant in an EVOO

To establish with greater certainty which adulterant was mixed with EVOO, and by how much, required the development of independent PLS1 calibration models based on gravimetrically prepared mixtures of authentic EVOO and adulterants. It was concluded that it was unlikely that a single FT-NIR model could be easily prepared that would determine the presence of different edible oils in EVOO. However, after examining the FT-NIR spectra of all mixtures of adulterants (Table 1) with EVOO (Table 2). It was concluded that four unique PLS1 calibration models could be generated for oils characterized by specific FA profiles.

To identify and quantify adulteration of EVOO therefore requires an examination of the pattern of predicted concentrations of the unknown oil obtained with all four PLS1 calibration models. Based on slope rules, and the like, the predicted pattern of adulterant concentrations is matched to one of the four groups of adulterants as shown in Table 3.

Specifically, the predicted nature and amount of an adulterant can be determined based on four simple, highly characteristic, and unique quantitative rules listed in Table 3. All authentic EVOO samples should show low or negative values with all four models, especially using the Model RO designed for refined olive oil. On the other hand, it is not possible to differentiate among the different oils within the high LA group or within the high OA group, since each group of oils shows the same slope responses when analyzed with the four PLS1 models.

The characteristic feature of an authentic EVOO is a high FT-NIR Index, a FA composition within the expected ranges for olive oils, and always associated with small or negligible slopes with all four PLS1 models, EVOO products should be analyzed upon receipt, since a low FT-NIR Index value may be due to several factors related to shipping and handling.

Further complication can arise if adulteration is more complex i.e., more than one adulterant is used to disguise adulteration. For example, a combination of hazelnut oil and refined oil or a palm olein and corn oil. In this case more comprehensive PLS1 calibration models would be required as we have demonstrated in this invention. Several models of 2 adulterants as well as 3 adulterants were created and used in this analysis. In Table 4, analysis of various commercial EVOO samples was conducted in which the number and type of adulterant added was analysed. The samples were analysed using a single adulterant, double adulterant and triple adulterant calibration model, and the results provided high FT-NIR index values for pure EVOO, while providing lower index values for adulterated oils.

### Confirmation of Adulterant and Amount Present Using the Standard Addition Technique

The standard addition technique was previously successfully used to accurately determine low trans FA levels in fat and oils. In the present study only when the correct adulterant was identified and its concentration predicted as described above, did the standard addition technique provide confirmatory evidence. In a standard addition experiment, if the resulting linear regression function exhibits a positive V-intercept, then the magnitude of this intercept would represent the concentration of the suspected adulterant that was originally present in the EVOO product (FIG. 10). The value of the Y-intercept should be very close in magnitude to the amount predicted by the PLS1 model that corresponds to the suspected adulterant. The lower limit of quantification is given by the RMSECV for each of the four models (see Table 2). None of the slope rules would apply to an authentic, unadulterated EVOO product, however if tested by the standard addition technique, it will result in a zero or negligible intercept with all four quantitative PLS1 models.

Thus, it is apparent that there has been provided, in accordance with the present invention, an analytical technique for the testing of an edible oil, and an EVOO in particular, which fully satisfies the goals, objects, and advantages set forth hereinbefore. Therefore, having described specific embodiments of the present invention, it will be understood that alternatives, modifications and variations thereof may be suggested to those skilled in the art, and that it is intended that the present specification embrace all such alternatives, modifications and variations as fall within the scope of the appended claims.

Additionally, for clarity and unless otherwise stated, the word “comprise” and variations of the word such as “comprising” and “comprises”, when used in the description and claims of the present specification, is not intended to exclude other additives, components, integers or steps. Further, the invention illustratively disclosed herein suitably may be practiced in the absence of any element which is not specifically disclosed herein. Also, unless otherwise specifically noted, all of the features described herein may be combined with any of the above aspects, in any combination.

Moreover, words such as “substantially” or “essentially”, when used with an adjective or adverb is intended to enhance the scope of the particular characteristic; e.g., substantially planar is intended to mean planar, nearly planar and/or exhibiting characteristics associated with a planar element. Further, use of the terms “he”, “him”, or “his”, is not intended to be specifically directed to persons of the masculine gender, and could easily be read as “she”, “her”, or “hers”, respectively. Also, while this discussion has addressed prior art known to the inventor, it is not an admission that all art discussed is citable against the present application.

TABLE 1
Typical FA concentrations (as % of total
FA) of selected plant oils analyzed by GC

| | 16:0 | 18:0 | 18:1n-9 | 18:2n-6 | 18:3n-3 |
|---|---|---|---|---|---|
| Extra virgin olive oil | 11.8 | 2.9 | 69.6 | 10.1 | 0.7 |
| Soybean oil | 10.1 | 4.6 | 24.2 | 50.2 | 6.8 |
| Sunflower oil | 6.6 | 3.4 | 28.0 | 59.3 | 0.1 |
| Corn oil | 11.6 | 2.0 | 28.5 | 54.2 | 1.4 |
| Canola oil | 4.1 | 1.8 | 59.9 | 19.4 | 6.9 |
| Hazelnut oil | 6.3 | 2.8 | 76.2 | 12.1 | 0.2 |
| High oleic acid safflower oil | 5.4 | 1.9 | 73.8 | 16.2 | 0.2 |
| Peanut oil | 10.2 | 2.8 | 53.7 | 25.5 | 0.1 |
| Palm olein | 37.3 | 4.1 | 43.0 | 11.7 | 0.2 |
| Refined olive oil | 12.8 | 3.1 | 70.2 | 8.3 | 0.7 |

TABLE 2
FT-NIR index, predicted adulterant amount, and FA composition for EVOO spiked with different concentrations of nine potential
adulterant oils: Soybean, sunflower, corn, cnnola, hazelnut, high oleic (HO) safflower, peanut and refined olive oil and palm olein.

| | | Predicted % adulterant oil added to EVOO | | | | | | | | | | |
|---|---|---|---|---|---|---|---|---|---|---|---|---|
| | FT-NIR | % | % | Model | Model | Model | Model | % Predicted FA (as % of total FA) | | | | |
| Oil mixtures | Index | Added | Predicted | LA* | OA* | PO* | RO* | 16:0 | 18:0 | 18:1n-9 | 18:2n-6 | 18:3n-3 |
| Accepted Range for FA (%) | | | | | | | | 7.5-20 | 0.5-5.0 | 55-83 | 3.5-21 | 0-1.5 |
| RMSECV** | 1.7 | | | 0.9 | 2.2 | 1 | 3.7 | 0.8 | 0.4 | 1.3 | 1.3 | 0.5 |
| EVOO | 93.9 | 0.0 | 1.0 | 1.0 | −15.4 | 6.7 | −7.6 | 13.2 | 2.3 | 71.8 | 7.0 | 0.4 |
| EVOO and soybean oil | 88.2 | 4.1 | 4.4 | 4.4 | 3.1 | 1.8 | −9.5 | 12.3 | 2.9 | 67.8 | 9.2 | 0.3 |
| EVOO and soybean oil | 79.4 | 7.9 | 7.9 | 7.9 | 15.9 | −2.1 | 7.5 | 12.5 | 2.2 | 68.4 | 9.4 | 0.8 |
| EVOO and soybean oil | 75.1 | 11.4 | 11.2 | 11.2 | 27.8 | −6.0 | 7.8 | 12.4 | 2.6 | 65.5 | 10.1 | 0.9 |
| EVOO and soybean oil | 68.6 | 14.6 | 14.2 | 14.2 | 38.8 | −12.4 | 6.2 | 12.5 | 2.6 | 65.9 | 10.3 | 1.5 |
| EVOO and soybean oil | 62.6 | 19.8 | 20.1 | 20.1 | 65.0 | −19.9 | 6.0 | 11.3 | 2.9 | 61.4 | 12.6 | 1.8 |
| EVOO and soybean oil | 57.6 | 24.3 | 24.1 | 24.1 | 85.1 | −27.1 | 2.8 | 12.0 | 2.8 | 59.6 | 15.2 | 2.1 |
| EVOO and soybean oil | 56.2 | 27.4 | 27.7 | 27.7 | 100.7 | −32.5 | −0.4 | 11.9 | 3.4 | 59.7 | 16.7 | 2.5 |
| EVOO and soybean oil | 52.9 | 32.1 | 32.5 | 32.5 | 121.4 | −39.7 | 0.5 | 11.5 | 3.1 | 56.2 | 18.7 | 2.5 |
| Soybean oil | 46.59 | 100.0 | 94.5 | 94.5 | 426.4 | −149.9 | −115.6 | 9.8 | 3.2 | 21.0 | 57.7 | 6.7 |
| EVOO | 96.6 | 0 | −0.5 | −0.5 | 0.1 | −4.4 | −23.3 | 14.1 | 2.6 | 64.9 | 10.6 | 0.0 |
| EVOO and sunflower oil | 87 | 3 | 3.3 | 3.3 | 15.6 | −6.9 | −4.5 | 13.8 | 2.5 | 63.8 | 11.5 | −0.2 |
| EVOO and sunflower oil | 79.3 | 5.9 | 6.5 | 6.5 | 25.1 | −12.3 | 0.6 | 14.0 | 2.2 | 61.3 | 13.1 | 0.3 |
| EVOO and sunflower oil | 72.5 | 8.6 | 8.9 | 8.9 | 38.4 | −15 | 12.1 | 14.0 | 2.4 | 61.4 | 13.6 | 0.4 |
| EVOO and sunflower oil | 68.2 | 11.1 | 11.1 | 11.1 | 48.3 | −17.6 | 21.3 | 13.3 | 2.1 | 63.7 | 14.2 | 0.9 |
| EVOO and sunflower oil | 58.9 | 13.5 | 13.9 | 13.9 | 59.8 | −22.2 | 21.4 | 13.5 | 2.2 | 61.8 | 15.1 | 0.7 |
| EVOO and sunflower oil | 53.8 | 15.8 | 16.4 | 16.4 | 74.1 | −24 | 28.3 | 12.6 | 2.3 | 58.4 | 16.6 | 0.6 |
| EVOO and sunflower oil | 50.5 | 18 | 18.5 | 18.5 | 86.2 | −27.7 | 29.6 | 12.1 | 2.3 | 58.3 | 17.4 | 0.7 |
| EVOO and sunflower oil | 46.9 | 20 | 19.8 | 19.8 | 93.2 | −31.9 | 28.7 | 12.2 | 1.9 | 56.5 | 18.7 | 0.4 |
| Sunflower oil | 45.8 | 100 | 91 | 91 | 426.8 | −164.2 | −135.6 | 9.5 | 2.4 | 27.5 | 57.5 | 2.1 |
| EVOO | 87.5 | 0 | 0.2 | 0.2 | 2.7 | −2.1 | −8.1 | 15.1 | 2.7 | 67.8 | 10.5 | 0.5 |
| EVOO and corn oil | 78.7 | 3.8 | 3.5 | 3.5 | 16.3 | −8 | −1.4 | 14.4 | 2.6 | 67.2 | 11.2 | 0.7 |
| EVOO and corn oil | 71.9 | 7.3 | 7.8 | 7.8 | 29.8 | −12.2 | 4.4 | 14.0 | 2.9 | 64.5 | 12.6 | 0.3 |
| EVOO and corn oil | 65.3 | 10.6 | 10.4 | 10.4 | 40.9 | −16.5 | 14 | 14.4 | 2.8 | 62.7 | 13.6 | 0.5 |
| EVOO and corn oil | 57.5 | 13.6 | 13.9 | 13.9 | 57.9 | −21.2 | 21.7 | 13.5 | 2.5 | 62.2 | 14.3 | 0.9 |
| EVOO and corn oil | 55.3 | 16.4 | 17 | 17 | 65.5 | −26.5 | 18 | 14.7 | 3.3 | 58.5 | 16.1 | 0.3 |
| EVOO and corn oil | 48.4 | 19.1 | 19.1 | 19.1 | 76.3 | −30.8 | 20 | 13.0 | 3.0 | 59.1 | 16.9 | 0.9 |
| EVOO and corn oil | 45.7 | 21.6 | 21.8 | 21.8 | 87.1 | −33.4 | 23.5 | 13.0 | 2.9 | 57.9 | 17.8 | 0.7 |
| EVOO and corn oil | 41.6 | 23.9 | 23.3 | 23.3 | 100.8 | −35.2 | 32.7 | 12.1 | 2.8 | 57.9 | 18.6 | 0.8 |
| Corn oil | 30.2 | 100 | 85.6 | 85.6 | 383 | −142.4 | −81 | 9.8 | 3.1 | 31.0 | 51.4 | 3.9 |
| EVOO | 83.7 | 0 | 2.1 | 2.1 | 5.4 | 5.3 | 4.7 | 14.3 | 3.1 | 66.0 | 10.1 | 0.4 |
| EVOO and canola oil | 84.4 | 3.5 | 4.8 | 4.8 | 16.4 | 1.2 | 3.8 | 13.7 | 2.5 | 66.2 | 10.2 | 1.0 |
| EVOO and canola oil | 74.3 | 6.8 | 7 | 7 | 24.9 | −2.3 | 14.2 | 13.8 | 2.7 | 61.9 | 11.9 | 0.5 |
| EVOO and canola oil | 66.2 | 9.8 | 9.7 | 9.7 | 33.1 | −4.6 | 22.2 | 13.1 | 3.0 | 62.9 | 10.6 | 1.4 |
| EVOO and canola oil | 59.5 | 12.7 | 12.1 | 12.1 | 39.5 | −7.3 | 30.6 | 11.6 | 2.4 | 63.9 | 10.7 | 1.6 |
| EVOO and canola oil | 54.1 | 15.3 | 14.7 | 14.7 | 48.8 | −8.8 | 35.7 | 11.7 | 2.5 | 63.8 | 11.0 | 1.4 |
| EVOO and canola oil | 52.6 | 17.9 | 16.5 | 16.5 | 55.3 | −12.7 | 28.4 | 12.5 | 2.5 | 61.1 | 11.6 | 1.6 |
| EVOO and canola oil | 49.2 | 20.2 | 17.3 | 17.3 | 59.3 | −13 | 31.1 | 12.6 | 2.6 | 62.7 | 12.1 | 1.4 |
| EVOO and canola oil | 50.1 | 22.5 | 18.3 | 18.3 | 66.8 | −16.4 | 23.7 | 12.2 | 2.9 | 62.9 | 12.3 | 1.5 |
| Canola oil | 41.8 | 100 | 60.2 | 60.2 | 247.5 | −93.3 | −54.2 | 5.5 | 1.9 | 55.2 | 25.3 | 6.1 |
| EVOO | 86 | 0.0 | 1.2 | 4.9 | 1.2 | −0.7 | 2.1 | 13.0 | 2.5 | 67.4 | 9.2 | 0.0 |
| EVOO and hazelnut oil | 77.9 | 4.1 | 5.1 | 6.9 | 5.1 | −2.4 | 7.1 | 12.7 | 2.0 | 65.7 | 9.1 | 0.6 |
| EVOO and hazelnut oil | 75.4 | 7.8 | 6.6 | 6.4 | 6.6 | −3.4 | 12.2 | 11.7 | 2.1 | 67.5 | 9.8 | 0.3 |
| EVOO and hazelnut oil | 70.5 | 11.3 | 9 | 7.2 | 9.0 | −5.7 | 17.2 | 12.0 | 2.2 | 67.6 | 9.2 | 0.5 |
| EVOO and hazelnut oil | 65.7 | 14.5 | 15.9 | 8.0 | 15.9 | −6.3 | 19.0 | 20.8 | 2.5 | 65.9 | 9.4 | 0.6 |
| EVOO and hazelnut oll | 61.7 | 19.6 | 21.8 | 8.7 | 21.8 | −8.2 | 24.1 | 12.4 | 2.8 | 65.7 | 10.3 | 0.3 |
| EVOO and hazelnut oil | 57.7 | 24.1 | 25 | 10.3 | 25.1 | −10.8 | 29.3 | 11.5 | 2.5 | 66.8 | 10.7 | 0.2 |
| EVOO and hazelnut oil | 53.9 | 31.8 | 30.7 | 13.1 | 30.7 | −14.5 | 33.1 | 10.0 | 1.7 | 67.6 | 10.4 | 0.4 |
| Hazelnut oil | 51.42 | 100.0 | 97.2 | 22.4 | 103.6 | −51.1 | 37.0 | 8.5 | 2.0 | 66.5 | 16.8 | 0.1 |
| EVOO | 85.1 | 0 | −10 | −0.9 | −10.3 | 4.1 | 3.5 | 13.7 | 2.1 | 73.3 | 6.6 | 0.5 |
| EVOO and peanut oil | 79.2 | 4.1 | 2.5 | 0.9 | 2.5 | 2.1 | 10.2 | 14.7 | 2.3 | 68.2 | 8.1 | −0.2 |
| EVOO and peanut oil | 68.4 | 7.9 | 7.2 | 0.9 | 7.2 | 3.2 | 22.8 | 14.5 | 2.4 | 68.2 | 8.5 | 0 |
| EVOO and peanut oil | 63.5 | 12.3 | 10 | 2.4 | 10.1 | 2.4 | 34.6 | 14.5 | 2.2 | 70.2 | 8.2 | 0.3 |
| EVOO and peanut oil | 59.6 | 15.5 | 13.4 | 1.4 | 13.4 | 4.1 | 34.2 | 14.9 | 2.3 | 69.4 | 8.8 | 0.4 |
| EVOO and peanut oil | 54 | 20.6 | 18.5 | 4.4 | 18.5 | −1.1 | 42.3 | 13.8 | 2 | 67.4 | 9.6 | 0.7 |
| EVOO and peanut oil | 49 | 25.1 | 25 | 5 | 25 | −3.2 | 48.1 | 14.2 | 2.4 | 67 | 10.4 | 0.5 |
| EVOO and peanut oil | 43.9 | 29.2 | 31.3 | 6.2 | 31.3 | −3.9 | 53.1 | 13.4 | 2.4 | 65.4 | 11.6 | 0.5 |
| EVOO and peanut oil | 43.4 | 32.8 | 34.2 | 6.7 | 34.2 | −5.5 | 55 | 14 | 2.3 | 64.3 | 12.5 | 0.7 |
| Peanut oil | 42.1 | 100 | 96.9 | 15 | 96.9 | −30.1 | 37.8 | 15.3 | 4.8 | 54.7 | 23.1 | 1.1 |
| EVOO | 91.3 | 0 | −3.6 | 2.4 | −3.6 | −2.5 | −2.2 | 14.1 | 2.4 | 70.2 | 9.3 | 0.7 |
| EVOO and HO safflower oil | 85.2 | 4.2 | 4.6 | 2.9 | 4.6 | −5.5 | 2.4 | 13.4 | 2.1 | 67.6 | 10.6 | 0.4 |
| EVOO and HO safflower oil | 83.6 | 8 | 7 | 3.6 | 7 | −6.7 | 3.3 | 13.4 | 2.2 | 66.9 | 10.4 | 0.1 |
| EVOO and HO safflower oil | 80.4 | 11.6 | 11.6 | 4.2 | 11.6 | −7.3 | 2.4 | 13.0 | 1.7 | 68.9 | 10.0 | 0.3 |
| EVOO and HO safflower oil | 78.1 | 14.8 | 14.1 | 5.8 | 14.1 | −9.2 | 4.8 | 12.2 | 2.0 | 70.4 | 10.0 | 0.7 |
| EVOO and HO safflower oil | 74.1 | 20.1 | 21.5 | 7.1 | 21.5 | −12.1 | 4.6 | 12.3 | 1.9 | 68.7 | 10.7 | 0.7 |
| EVOO and HO safflower oil | 71.9 | 24.7 | 24.7 | 7.6 | 24.7 | −13.7 | 3.1 | 12.3 | 2.0 | 68.2 | 11.1 | 0.4 |
| EVOO and HO safflower oil | 69.8 | 28.8 | 30.4 | 9 | 30.4 | −17 | 11.3 | 12.2 | 2.1 | 67.5 | 11.3 | 0.3 |
| EVOO and HO safflower oil | 69.6 | 32.5 | 35.4 | 10.5 | 35.4 | −17.9 | 6.7 | 11.6 | 1.9 | 68.0 | 11.8 | 0.4 |
| HO safflower oil | 45.1 | 100 | 120.1 | 25.5 | 120.1 | −52.9 | 16.8 | 7.4 | 1.5 | 71.8 | 17.2 | 0.0 |
| EVOO | 85.7 | 0.0 | 0.0 | 0.8 | −0.5 | −2.5 | 7.9 | 13.7 | 1.6 | 67.1 | 10.6 | 0.1 |
| EVOO and palm olein | 73.1 | 4.1 | 4.3 | −0.3 | −6.1 | 3.8 | 26.1 | 14.1 | 2.7 | 66.0 | 10.2 | −0.1 |
| EVOO and palm olein | 65.7 | 7.9 | 7.9 | −1.6 | −9.8 | 8.0 | 43.3 | 14.9 | 2.3 | 63.5 | 9.7 | −0.1 |
| EVOO and palm olein | 61.7 | 11.4 | 11.5 | −2.5 | −19.0 | 11.4 | 50.1 | 15.0 | 2.2 | 63.1 | 9.5 | −0.2 |
| EVOO and palm olein | 56.1 | 14.5 | 14.7 | −3.1 | −24.3 | 14.3 | 58.5 | 15.7 | 2.5 | 62.1 | 9.5 | 0.2 |
| EVOO and palm olein | 51.2 | 20.5 | 20.5 | −5.1 | −32.8 | 20.7 | 81.0 | 16.3 | 2.7 | 62.1 | 9.5 | 0.2 |
| EVOO and palm olein | 45.7 | 25.5 | 25.3 | −6.3 | −41.4 | 25.8 | 86.9 | 17.8 | 2.7 | 60.9 | 9.3 | 0.5 |
| Palm olein | 27.77 | 100.0 | 93.2 | −33.2 | −173.9 | 96.6 | 157.5 | 37.3 | 4.1 | 43.0 | 11.7 | 0.2 |
| EVOO | 86 | 0.0 | −10.0 | 3.8 | −1.1 | −6.6 | −10.0 | 10.6 | 1.9 | 72.7 | 6.8 | 0.7 |
| EVOO and refined olive oil | 76.5 | 4.1 | 2.8 | 4.3 | 2.6 | −6.7 | 2.8 | 11.9 | 1.9 | 72.6 | 5.9 | 0.8 |
| EVOO and refined olive oil | 75 | 7.9 | −0.8 | 4.7 | −0.9 | −5.2 | −0.8 | 13.0 | 2.3 | 71.9 | 5.5 | 0.5 |
| EVOO and refined olive oil | 69.3 | 11.5 | 12.1 | 4.5 | −2.2 | −4.9 | 12.1 | 12.5 | 2.0 | 73.5 | 5.1 | 0.8 |
| EVOO and refined olive oil | 67.1 | 14.7 | 16.5 | 6.2 | 4.3 | −5.0 | 16.5 | 12.4 | 1.7 | 72.0 | 5.3 | 1.1 |
| EVOO and refined olive oil | 64.2 | 19.9 | 21.7 | 5.0 | 0.0 | −4.4 | 21.7 | 12.2 | 2.2 | 73.8 | 5.7 | 0.8 |
| EVOO and refined olive oil | 61.8 | 24.5 | 27.1 | 4.5 | 2.6 | −3.5 | 27.1 | 12.1 | 2.4 | 73.6 | 5.9 | 0.5 |
| EVOO and refined olive oil | 58.7 | 32.3 | 32.4 | 5.3 | 1.5 | −2.7 | 32.4 | 12.6 | 2.1 | 72.5 | 6.1 | 0.9 |
| EVOO and refined olive oil | 56.5 | 41.7 | 42.3 | 3.9 | 3.9 | 2.0 | 42.3 | 12.8 | 2.5 | 71.4 | 6.7 | 0.3 |
| EVOO and refined olive oil | 54.6 | 51.7 | 50.3 | 3.1 | 1.7 | −2.0 | 50.3 | 12.6 | 3.1 | 70.4 | 7.7 | 0.3 |
| EVOO and refined olive oil | 51.7 | 60.4 | 56.2 | 4.7 | 1.4 | −1.6 | 56.2 | 12.6 | 3.1 | 70.6 | 7.5 | 0.0 |
| Refined olive oil | 38.4 | 100.0 | 96.3 | 3.7 | 0.1 | 1.1 | 96.3 | 12.8 | 3.1 | 70.2 | 8.3 | 0.7 |

TABLE 3
Slope rules for predicting nature and concentration of an adulterant oil in EVOO

| | | | | | Rules Based on the | |
|---|---|---|---|---|---|---|
| | Magnitude and Sign of | | | | Magnitudes of Slopes | |
| | Linear Regression Function Slope for | | | | Derived from Models LA, | |
| | Plots of Predicted Adulterant Amount vs. True | | | | OA, PO, and RO | |
| | Adulterant Amount in EVOO | | | | Linear Regression Function | |
| Adulterating oil | Model LA, 1 | Model OA, 2 | Model PO, 3 | Model RO, 4 | Positive Slope | Negative Slope |
| Soybean* | 1.0 | 4.2 | −1.5 | n/a*** | 2 >> 1 | 3 |
| Sunflower* | 1.0 | 4.7 | −1.4 | n/a*** | 2 >> 1 | 3 |
| Corn* | 1.0 | 4.0 | −1.4 | n/a*** | 2 >> 1 | 3 |
| Canola* | 0.8 | 2.7 | −0.9 | n/a*** | 2 >> 1 | 3 |
| Hazelnut** | 0.2 | 1.0 | −0.4 | n/a*** | 2 > 1 | 3 |
| Peanut** | 0.2 | 1.2 | −0.3 | n/a*** | 2 > 1 | 3 |
| High Oleic Safflower** | 0.3 | 1.1 | −0.5 | n/a*** | 2 > 1 | 3 |
| Palm Olein | −0.3 | −1.6 | 1.1 | 3.1 | 4 >> 3 | 1, 2 |
| Refined olive | 0.0 | 0.04 | 0.1 | 1.1 | 4 >> 1, 2, 3 | |

*The group of high linoleic acid (18:2n-6) adulterant oils follow the same rule
**The group of high oleic acid (18:1n-9) adulterant oils follow the same rule
***Not applicable: Non-linear regression function was found with Model RO

TABLE 4
Table of Commercial samples

| | | FT-NIR | % | | Calibration Model |
|---|---|---|---|---|---|
| SampleNo. | Description | Index | adulterant | Type of Adulterant | Applied |
| M1466OM2 | Commercial Extra virgin Olive oil 1 | 97.1 | 0 | Extra Virgin Olive oil | All three |
| M1475OM2 | Commercial Extra virgin Olive oil 2 | 85.6 | 0 | Extra Virgin Olive oil | All three |
| M1433OM2 | Commercial Extra virgin Olive oil 3 | 87.3 | 0 | Extra Virgin Olive oil | All three |
| M1509OM2 | Commercial Extra virgin Olive oil 4 | 53.7 | 86.3 | Soybean | Single Adulterant |
| M1510OM2 | Commercial Extra virgin Olive oil 5 | 52.6 | 91 | Soybean | Single Adulterant |
| M1501OM2 | Commercial Extra virgin Olive oil 6 | 51.3 | 86.6 | Canola | Single Adulterant |
| M1468OM2 | Commercial Extra virgin Olive oil 7 | 60.2 | 66.9 | Refined oil | Single Adulterant |
| M1441OM2 | Commercial Extra virgin Olive oil 8 | 74.8 | 19.4 | Palm Olein and Soybean | Double adulterant |
| M1435OM2 | Commercial Extra virgin Olive oil 9 | 48.4 | 30.3 | Palm Olein and Soybean | Double adulterant |
| M1438OM2 | Commercial Extra virgin Olive oil 10 | 69.6 | 22.2 | Palm Olein and Soybean | Double adulterant |
| M1470OM2 | Commercial Extra virgin Olive oil 11 | 57 | 15.8 | Palm Olein and Soybean | Double adulterant |
| M1456OM2 | Commercial Extra virgin Olive oil 12 | 71.3 | 19.3 | Hazelnut and refined olive | Double adulterant |
| M1457OM2 | Commercial Extra virgin Olive oil 13 | 56 | 31.1 | Hazelnut, refined olive, corn | Triple adulterant |
| M1476OM2 | Commercial Extra virgin Olive oil 14 | 14.4 | 80.7 | Hazelnut, refined olive, corn | Triple adulterant |

What is claimed is:

1. A method based on FT-NIR spectroscopy to rapidly analyse an edible oil sample, and preferably an olive oil sample, and most preferably an Extra Virgin Olive Oil (“EVOO”) sample, to determine whether the sample has been adulterated, as herein described.

2. A method as claimed in claim 1, based on an FT-NIR spectroscopy based analytical technique to rapidly analyse an edible oil, and preferably an olive oil, and most preferably an EVOO, which can identify an oil adulterant, and preferably both identify and quantify, the level of adulteration, present in the oil sample.

3. A method as claimed in claim 1, based on an FT-NIR spectroscopy based analytical technique which can identify and quantify at least 1 adulterant present in an olive oil sample, and preferably an EVOO sample.

4. A method as claimed in claim 3, which can be used to identify between 1 and 6 adulterants, and preferably between 1 and 3 adulterants, are present in the oil sample being analysed.

5. A method for the detection of adulteration in an edible oil, and preferably an olive oil sample, and most preferably, an EVOO sample, comprising:
preparing a calibration model for said edible oil, to be tested;
conducting an FT-NIR spectroscopy analysis of an unknown sample;
analysing the FT-NIR spectroscopy analysis of said unknown sample, at selected wave numbers; and,
comparing the FT-NIR spectroscopy analysis of said unknown sample, to said calibration model of said edible oil, at said selected wave numbers, in order to determine whether said edible oil sample had been adulterated.

6. A method as claimed in claim 5, wherein the selected wave number is at, or essentially at, 5280 cm⁻¹ and/or 5180 cm⁻¹.

7. A method as claimed in claim 5 wherein the results of said comparison are used to prepare an FT-NIR index, based on the results observed at the selected wave number which shows the low boiling (volatile compound) have been affected, or otherwise reduced.

8. A method as claimed in claim 7 wherein said FT-NIR index is based on the ratio of the adsorption values observed at 5280 cm⁻¹ and 5180 cm⁻¹ for any given oil or adulterated blend.

9. A method as claimed in claim 8 wherein the FT-NIR index is at its highest (e.g. 100%) when pure EVOO is tested, and wherein FT-NIR indices of over 80% are preferred, and levels of over 90, 95 or even more 99%, are more preferred.

10. A method as claimed in claim 1, additionally comprising, once adulteration of the edible oil sample, has been determined, the determination of the type and/or quantity of the level of adulteration in an adulterated edible oil sample, and preferably an adulterated olive oil sample, and most preferably, an adulterated EVOO sample, comprising:
preparing an FT-NIR calibration matrix based on calibration models based on said edible oil, having at least one, and preferably up to 4, and even more preferably up to 6 adulterants;
analysing said adulterated edible oil using an FT-NIR spectroscopy based technique to produce an FT-NIR oil analysis;
comparing said FT-NIR oil analysis with said FT-NIR calibration matrix, to determine the type of adulterant, the level of adulterant present, or determine both the type and level of adulterant or adulterants, present in said sample.
